(19) United States
(12) Patent Application Publication
(10) Pub. No.: US 2025/0084349 A1
(43) Pub. Date: Mar. 13, 2025

(54) RAW MATERIAL COMPOSITIONS WITH REDUCING SUGARS AND MAILLARD REACTION INHIBITING ENZYMES

(71) Applicant: HENKEL AG & CO. KGAA, Düsseldorf (DE)

(72) Inventor: Daniel Thomas PIORKOWSKI, Fairfield, CT (US)

(21) Appl. No.: 18/828,617

(22) Filed: Sep. 9, 2024

Related U.S. Application Data
(60) Provisional application No. 63/537,064, filed on Sep. 7, 2023.

(51) Int. Cl. C11D 3/386; C11D 1/72; C11D 3/22; C11D 3/30; C11D 3/43; C12N 9/04

(52) U.S. Cl. CPC C11D 3/38654; C11D 1/72; C11D 3/221; C11D 3/222; C11D 3/30; C11D 3/43; C12N 9/0006; C12Y 101/03005

(57) ABSTRACT

Methods of preventing browning reactions of a raw material compositions, and a formulations containing a sustainable raw material useful as a component in liquid laundry detergents are described. The methods and formulations involve adding a Maillard reaction inhibiting enzyme to the raw material composition, which prevents the reducing sugar from reacting with an amine source and causing discoloration. The raw material composition can be used as a component in a detergent composition, such as a liquid laundry detergent, that exhibits increased shelf-life and consumer confidence. The sustainable raw material can be a sustainable polymer, a sustainable solvent, or a mixture thereof, and the Maillard reaction inhibiting enzyme can be hexose oxidase, fructosamine oxidase, fructosamine kinase, carbohydrate oxidase, or combinations thereof.

### CROSS-REFERENCE TO RELATED APPLICATION

This application claims the benefit of U.S. Provisional Application No. 63/537,064, filed Sep. 7, 2023, the entire contents of which are incorporated by reference herein.

### FIELD OF THE DISCLOSURE

The disclosure relates to raw material compositions for incorporation in detergent compositions, such as liquid laundry detergent compositions. The disclosure further relates to methods of preventing discoloration (for example, browning or yellowing) of raw material compositions, and formulation including raw material compositions having increased shelf-lives.

### BACKGROUND OF THE DISCLOSURE

In detergent compositions in both unit dose forms and non-unit dose forms, there is an ongoing trend to include sustainable raw materials therein. Sustainable raw materials are derived from renewable, recycled and waste carbon sources and their combination, which at the end of life can be recycled, biodegraded, or composted. Sustainable raw materials also exhibit reduced environmental impact throughout their life cycle. The use of sustainable raw materials within detergent compositions can reduce the cost of formulating the detergent composition and can provide an environmentally friendly product.

In some instances, the sustainable raw material used in formulating the detergent composition includes a reducing sugar, such as, for example, fructose or glucose. The reducing sugar can be a contaminant (i.e., residual) in the sustainable raw material or it can be part (i.e., a moiety) of the sustainable raw material, for example, a monomer of a polymer, which degrades from the sustainable raw material over time.

Reducing sugars undergo a Maillard reaction and cause browning in products when in the presence of amines and heat. In laundry detergents, amines can be present in the form of, for example, monoethanolamine (which is used as a neutralizer) or enzymes (e.g., proteins). Browning reactions cause detergent compositions to turn yellow over time, especially when stored at elevated temperatures such as, for example, from 35° C. to 50° C. In some cases, it has been observed that as little as 40 ppm of reducing sugars can cause browning reactions to occur within a detergent composition over time.

The discoloration of detergent compositions caused by the Maillard reaction can lead to consumer dissatisfaction and consumer apprehension that the detergent compositions are no longer useable. Thus, there is a need for providing laundry detergent compositions in which browning reactions can be prevented.

### SUMMARY OF THE DISCLOSURE

Various aspects of the disclosure are directed to methods of preventing a browning reaction of a raw material composition, and formulations containing a sustainable raw material useful as a component in a detergent composition, such as a liquid laundry detergent. Methods according to various aspects of the disclosure include adding an enzyme to a raw material composition containing a sustainable raw material that includes a reducing sugar. The enzyme inhibits the reducing sugar from subsequently reacting with an amine source and thus browning is prevented. The enzyme prevents a Maillard reaction from occurring and thus is referred to herein as a “Maillard reaction inhibiting enzyme”.

Formulations according to various aspects of the disclosure include a raw material composition containing a sustainable raw material including a reducing sugar and the Maillard reaction inhibiting enzyme. Formulations according to various aspects of the disclosure exhibit increased shelf-life and can be used in providing laundry detergent compositions that do not undergoing yellowing and thus exhibit increased shelf-lives compared to equivalent laundry detergent compositions that lack such an enzyme. Since yellowing is prevented, consumer confidence in detergent compositions containing formulations according to the disclosure will be improved.

In accordance with various aspects of the disclosure, methods of preventing a browning reaction of a raw material composition are provided. In some instances, such a method comprises adding a Maillard reaction inhibiting enzyme to a raw material composition, where the raw material composition comprises a sustainable raw material including a reducing sugar. In accordance with various aspects of the disclosure, the Maillard reaction inhibiting enzyme prevents the reducing sugar from reacting with an amine source. The amine source is typically used as a component of another composition (such as, for example, a detergent composition) to which the raw material composition containing the Maillard reaction inhibiting enzyme can be added thereto as one of the components of another composition.

In accordance with various aspects of the disclosure, the reducing sugar can be a residual contaminant present in the raw material composition, or it can be a moiety of the sustainable raw material that can be released upon degradation. Degradation of the reducing sugar can occur during formulation and storage, especially when the raw material composition is stored at elevated temperatures.

In some instances, the raw material composition consists essentially of a sustainable raw material and a reducing sugar. In some instances, the raw material composition consists of a sustainable raw material and a reducing sugar.

In some instances, the sustainable raw material comprises a sustainable polymer. In some embodiments, the sustainable raw material comprises a sustainable solvent. In some embodiments, the sustainable raw material comprises a mixture of a sustainable polymer and a sustainable solvent.

In some instances, when the sustainable raw material is a sustainable polymer, the sustainable polymer comprises a starch, a glycogen, a galactogen, a cellulose, a chitin or mixtures thereof.

In some instances, the reducing sugar is a reducing sugar monomer of the sustainable polymer.

In some instances, the reducing sugar monomer comprises fructose, glucose, hexose, lactose, glyceraldehyde, arabinose, maltose, galactose, ribose, xylose, cellobiose or combinations thereof.

In some instances, when the sustainable raw material is or comprises a sustainable solvent. The sustainable solvent can be or comprises sorbitol, xylitol, mannitol, lactitol, isomalt, maltitol, a hydrogenated starch hydrolysate, sucrose, stachyose, verbascose, trehalose, raffinose or combinations thereof.

In some instances, the reducing sugar is a reducing sugar of the sustainable solvent. In some instances, reducing sugar of the sustainable solvent can be or comprises fructose, glucose, hexose, lactose, glyceraldehyde, arabinose, maltose, galactose, ribose, xylose, cellobiose or combinations thereof.

In some instances, the Maillard reaction inhibiting enzyme is or comprises hexose oxidase, fructosamine oxidase, fructosamine kinase, carbohydrate oxidase or combinations thereof.

In some instances, the Maillard reaction inhibiting enzyme is added in an amount of from about 0.001 weight percent to about 3 weight percent of the raw material composition.

In some instances, raw material compositions according to the disclosure are produced from a fermentation reaction.

In another aspect of the disclosure, formulations are provided. In some instances, formulations according to various aspects of the disclosure comprise a raw material composition comprising a sustainable raw material including at least one reducing sugar; and a Maillard reaction inhibiting enzyme.

In some instances, formulations according to various aspects of the disclosure consist essentially of, or consist of, a raw material composition and a Maillard reaction inhibiting enzyme. In such instances, the raw material composition can comprise, consists essentially of, or consists of, the sustainable raw material and the reducing sugar.

In some instances, a sustainable raw material present in a formulation according to the disclosure comprises a sustainable polymer. In some instances, a sustainable raw material present in a formulation according to the disclosure comprises a sustainable solvent. In some instances, a sustainable raw material present in a formulation according to the disclosure comprises a mixture of a sustainable polymer and a sustainable solvent.

In some instances, in which the sustainable raw material includes a sustainable polymer, the sustainable polymer comprises a starch, a glycogen, a galactogen, a cellulose, a chitin or mixtures thereof.

In some instances, a reducing sugar of a sustainable polymer present in the formulation of the present disclosure is a reducing sugar monomer of the sustainable polymer.

In some instances, the reducing sugar monomer comprises fructose, glucose, hexose, lactose, glyceraldehyde, arabinose, maltose, galactose, ribose, xylose, cellobiose or combinations thereof.

In some instances, in which the sustainable raw material is or comprises a sustainable solvent, the sustainable solvent comprises sorbitol, xylitol, mannitol, lactitol, isomalt, maltitol, a hydrogenated starch hydrolysate, sucrose, stachyose, verbascose, trehalose, raffinose or combinations thereof.

In some instances, a reducing sugar of a sustainable solvent present in a formulation according to the disclosure comprises fructose, glucose, hexose, lactose, glyceraldehyde, arabinose, maltose, galactose, ribose, xylose, cellobiose or combinations thereof.

In some instances, the Maillard reaction inhibiting enzyme that is present in a formulation according to the disclosure comprises hexose oxidase, fructosamine oxidase, fructosamine kinase, carbohydrate oxidase or combinations thereof.

In some instances, the Maillard reaction inhibiting enzyme is present in a formulation according to the disclosure in an amount of from about 0.001 weight percent to about 3 weight percent.

In some instances, a formulation according to the disclosure can further include water. In some instances, formulations according to the disclosure can include water in an amount from about 1 weight percent to about 99 weight percent.

### BRIEF DESCRIPTION OF THE DRAWINGS

FIG. 1 is an image depicting the yellow pantone scale for the yellowing of detergent compositions from reducing sugar agents.

### DETAILED DESCRIPTION

The present disclosure will now be described in greater detail by referring to the following discussion and drawings that accompany the present disclosure. In the following description, numerous specific details are set forth, such as particular structures, components, materials, dimensions, processing steps and techniques, in order to provide an understanding of the various embodiments of the present disclosure. However, it will be appreciated by one of ordinary skill in the art that the various embodiments of the present disclosure may be practiced without these specific details. As used throughout the present disclosure, the term “about” generally indicates no more than ±10%, ±5%, ±2%, ±1% or ±0.5% from a number. When a range is expressed in the present disclosure as being from one number to another number (e.g., 20 to 40), the present disclose contemplates any numerical value that is within the range (i.e., 22, 24, 26, 28.5, 31, 33.5, 35, 37.7, 39 or 40) or any in amount that is bounded by any of the two values that can be present within the range (e.g., 28.5-35).

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

As stated above, a method of preventing a browning reaction of a raw material composition and a formulation containing a sustainable raw material useful as a component in a liquid laundry detergent are provided. The method of the present disclosure, which provides the formulation of the present disclosure, includes adding a Maillard reaction inhibiting enzyme to a raw material composition containing a sustainable raw material that includes a reducing sugar. The Maillard reaction inhibiting enzyme inhibits the reducing sugar from subsequently reacting with an amine source (typically present in another composition, e.g., a laundry detergent composition). The formulation of the present disclosure has increased shelf-life and can be used in providing a laundry detergent composition that does not undergoing yellowing and thus has an increased shelf-life compared to an equivalent laundry detergent composition that lacks such an enzyme. Since yellowing is prevented, consumer confidence in the detergent composition containing the formulation of the present disclosure would be improved. These and other aspects of the present disclosure will now be described in greater detail.

Any raw material composition that comprises, consists essentially of, or consists of, a sustainable raw material including a reducing sugar can be used in accordance with various aspects of the disclosure. The reducing sugar can be a residual contaminant present in the raw material composition, or it can be a moiety of the sustainable raw material that can be released upon degradation. Degradation of the reducing sugar can occur during formulation and storage, especially when the raw material composition is stored at elevated temperatures. In some instances, the reducing sugar is a monosaccharide. In some instances, the reducing sugar is galactose, glucose, glyceraldehyde, fructose, ribose, or xylose. In some instances, the raw material composition is incorporated in a detergent composition. In some instances, the detergent composition is a laundry detergent composition. In some instances, the reducing sugar is present in an amount ranging from about 0.01 to about 5 wt % of a detergent composition. In some instances, the reducing sugar is present in an amount ranging from about 0.0001 to about 7 wt % of a detergent composition. In some instances, the reducing sugar is present in an amount ranging from about 0.01 to about 5 wt % of a raw material composition. In some instances, the reducing sugar is present in an amount ranging from about 0.0001 to about 7 wt % of a raw material composition.

In some instances, the sustainable raw material comprises a sustainable polymer, a sustainable solvent or a mixture of a sustainable polymer and a sustainable solvent. In some instances, the sustainable raw material including the reducing sugar is made from a fermentation reaction.

When a sustainable polymer is used as a sustainable raw material, the sustainable polymer can be present in the raw material composition (and thus the formulation of the present disclosure) in an amount of from about 0.01 weight percent to about 10 weight percent. In some instances, the sustainable polymer can be present in the raw material composition (and thus the formulation of the present disclosure) in an amount of from about 0.25 weight percent to about 2 weight percent. In some instances, the sustainable polymer can be present in the raw material composition (and thus the formulation of the present disclosure) in an amount of from about 0.5 weight percent to about 1 weight percent.

In some instances, the sustainable polymer is a polysaccharide. A polysaccharide is a polymer made of chains of monosaccharides that are joined by glycosidic linkages. A polysaccharide typically consists of more than ten monosaccharide units. Polysaccharides may be linear or branched. Linear polysaccharides are typically rigid polymers, while branched polysaccharides are typically soluble in water. In accordance with various aspects of the disclosure, polysaccharides include starches, glycogens, galactogens, cellulose and/or chitin. An illustrative example of a polysaccharide that can be used in the present disclosure as the sustainable polymer includes, but is not limited to, a poly alpha-1,6-glucan.

As mentioned above, sustainable polymers according to the disclosure include reducing sugars which are in the form of reducing sugar monomers. A reducing sugar monomer can be a contaminate (residual) or it can be a moiety of the sustainable polymer that can be released upon degradation. In detergent compositions including an amine source, reducing sugar monomers (residual or released) and the amine source can react together under heat to cause unwanted yellowing of the detergent composition. In some instances, the reducing sugar monomer is present in the sustainable polymer in an amount of from about 0.0001 weight percent to about 1 weight percent, alternatively in an amount of from about 0.005 weight percent to about 0.5 weight percent. In some instances, the reducing sugar monomer is present in the sustainable polymer in an amount ranging from about 0.0001 to about 5 weight percent of the sustainable polymer. In accordance with various aspects of the disclosure, a reducing sugar monomer is any sugar monomer that can act as a reducing agent. In some instances, the reducing sugar monomer can include a monosaccharide or disaccharide. Exemplary reducing sugar monomers that can be contained in a sustainable polymer include, but are not limited to, fructose, glucose, hexose, lactose, glyceraldehyde, arabinose, maltose, galactose, ribose, xylose, cellobiose or combinations thereof.

When a sustainable solvent is used as a sustainable raw material, the sustainable solvent can be present in the raw material composition (and thus the formulation of the present disclosure) in an amount of from about 0.01 weight percent to about 60 weight percent of the raw material composition. In some instances, the sustainable solvent can be present in the raw material composition (and thus the formulation of the present disclosure) in an amount of from about an amount of from about 1 weight percent to about 30 weight percent of the raw material composition. In some instances, the sustainable solvent can be present in the raw material composition (and thus the formulation of the present disclosure) in an amount of from about an amount of from about 5 weight percent to about 20 weight percent of the raw material composition.

As mentioned above, the sustainable solvents according to various aspects of the disclosure include a reducing sugar. The reducing sugar can be a contaminate (residual) or it can be a moiety of the sustainable solvent that can be released upon degradation. In detergent compositions including an amine source, the reducing sugar (residual or released) and the amine source can react together under heat to cause unwanted yellowing of the detergent composition. In some instances, the reducing sugar is present in the sustainable solvent in an amount of from about 0.0001 weight percent to about 1 weight percent, alternatively in an amount of from about 0.005 weight percent to about 0.5 weight percent. A reducing sugar is any sugar that can act as a reducing agent. In some instances, the reducing sugar can include a monosaccharide or disaccharide. Exemplary reducing sugars that can be contained in the sustainable solvent include, but are not limited to, fructose, glucose, hexose, lactose, glyceraldehyde, arabinose, maltose, galactose, ribose, xylose, cellobiose or combinations thereof.

In accordance with various aspects of the disclosure, the Maillard reaction inhibiting enzyme is any enzyme that can prevent a browning reaction from occurring. That is, the Maillard reaction inhibiting enzyme can inhibit the reaction (i.e., Maillard reaction) between a reducing sugar of a sustainable raw material and an amine source (that is typically present in another composition such as, for example, a laundry detergent composition). The Maillard reaction inhibiting enzyme is added to the raw material composition (and thus is contained in a formulation according to the disclosure) in an amount of from about 0.001 weight percent to about 3 weight percent, alternatively in an amount of from about 0.005 weight percent to about 0.5 weight percent, and alternatively in an amount from about 0.01 weight percent to about 0.1 weight percent. In some instances, the Maillard reaction inhibiting enzyme is added to the raw material composition in an amount from about 0.001 weight percent to about 5 weight percent of the raw material composition. In some instances, the Maillard reaction inhibiting enzyme is a reducing sugar oxidation enzyme. Such oxidation enzymes oxidize the reducing sugar that is present and prevent subsequent yellowing of a detergent composition from occurring. In some instances, the Maillard reaction inhibiting enzyme is an enzyme that reacts with fructose and glucose since they are commonly used in producing sustainable raw materials. Illustrative examples of Maillard reaction inhibiting enzymes that can used in accordance with various aspects of the disclosure include, but are not limited to, hexose oxidase, fructosamine oxidase, fructosamine kinase, or carbohydrate oxidase. It is noted that within the range mentioned above, the Maillard reaction inhibiting enzyme can prevent the Maillard reaction from occurring throughout the entire shelf-life of a formulation according to the disclosure and throughout the entire shelf-life of a detergent composition according to the disclosure that includes such a formulation as one of its components.

The Maillard reaction inhibiting enzyme can be added to a raw material composition that contains at least one of the sustainable raw materials mentioned above utilizing techniques that are well known to those skilled in the art. In some instances, the addition includes metered addition. The addition occurs with continuous stirring and is typically performed at room temperature, e.g., from about 20° C. to about 30° C. This addition provides the formulation in accordance with the present application. In addition to the Maillard reaction inhibiting enzyme and the raw material composition mentioned above, formulations according to the disclosure can include additional components as described below. The additional components can be present in a raw material composition that includes a sustainable raw material and a reducing sugar, or they can be added after forming the raw material composition.

In some instances, formulations according to the disclosure are aqueous formulations that include water. In instances where formulations include water, the water is typically present in an amount from about 1 weight percent to about 99 weight percent, alternatively in an amount from about 20 weight percent to about 70 weight percent. In some instances, the water is present in an amount ranging from about 30 weight percent to about 60 weight percent of the formulation.

In some instances, formulations according to the disclosure are non-aqueous formulations. In such instances, a non-aqueous formulation includes a solvent other than water. For example, an alcohol, ether, and/or ester can be used in providing a non-aqueous formulation according to the disclosure. In instances where the formulation is non-aqueous, the solvent other than water is typically present in an amount from about 1 weight percent to about 99 weight percent, alternatively in an amount from about 20 weight percent to about 70 weight percent. In some instances, the non-aqueous formulation is present in an amount ranging from about 30 weight percent to about 60 weight percent of the formulation.

Formulations according to the disclosure can also include at least one impurity, a pH adjusting agent, a preservative, or any combination thereof. When at least one impurity is present, the at least one impurity can be present in a formulation in an amount of from about 0.2 weight percent or less, alternatively in an amount of about 1 ppm or less. Illustrative examples of impurities that can be present include, but are not limited to, alkylene glycols (such as, for example, diethylene glycol and/or ethylene glycol), metals (such as, for example, Ni, Pt, and/or Pd), and any combination thereof. Generally, the at least one impurity is not intentionally added to the formulation, but instead can be a leftover material that is present in the raw material composition.

When a pH adjusting agent (or buffer) is present, the pH adjusting agent can be present in a formulation in an amount of from about 1 weight percent or less. Illustrative examples of pH adjusting agents that can be present in a formulation according to the disclosure include, but are not limited to, acids and/or bases. Illustrative acids that can be used include, but are not limited to, citric acid, and/or acetic acid. In some instances, the acid can be, for example, lactic acid, stearic acid, palmitic acid or oleic acid.

In some instances, the pH adjusting agent is a base. Illustrative bases that can be used include, but are not limited to, KOH and/or NaOH. In some instances, the base is calcium oxide. When present, the pH adjusting agent is employed to adjust the formulation containing the raw material composition to a desired pH. In some cases, the pH adjusting agent can also affect the stability, efficacy and performance of other components present in the formulation. The pH adjusting agent can be added utilizing techniques that are well known to those skilled in the art. The formulation of the present disclosure typically has a pH from 6 to 12, alternatively a pH from 6.5 to 10.

When a preservative is present, the preservative can be present in a formulation in an amount of from about 1 weight percent or less. The preservative can prevent undesirable chemical changes of the raw material composition or prevent degradation by bacteria of the raw material composition that is present in the formulation. Illustrative examples of preservatives that can be employed in a formulation according to the disclosure include, but are not limited to, antimicrobial preservatives. Antimicrobial preservatives are preservatives that prevent degradation by bacteria and include, for example, isothiazolinones. Benzisothiazolinone (BIT), methylchloroisothiazolinone (CIT) and methylisothiazolinone (MIT) are illustrative types of isothiazolinones that can be used as a preservative. In some instances, the preservative is one or more of a microbiocide, an algicide, and a fungicide. In some instances, the preservative is a mixture of 5-chloro-2-methyl-2H-isothiazol-3-one and 2-methylisothiazol-3(2H)-one and 1,2-benzisothiazol-3(2H)-one. In some instances, preservative is methylisothiazolinone, chloromethylisothiazolinone, benzisothiazolinone, sorbic acid, sodium benzoate, formaldehyde, borate, and glutaraldehyde. In some instances, detergent compositions according to various aspects of the disclosure may be free of, or substantially free of, preservatives The preservative can be added utilizing techniques that are well known to those skilled in the art.

In some instances, formulations according to the disclosure can be used as a component of a detergent composition. The detergent composition can be in a unit dose form or anon-unit dose form. The inclusion of a formulation according to the disclosure to a detergent composition (in either a unit dose or a non-unit dose form) provides increased shelf-life to the detergent composition. In addition to a formulation according to the disclosure, a detergent composition includes at least one surfactant, an amine source that can react with a reducing sugar present in the formulation, and water. When the detergent composition includes a formulation according to the disclosure as one of its components, the Maillard reaction inhibiting enzyme in the formulation of the present disclosure inhibits the Maillard reaction between the reducing sugar and the amine source and thus the detergent composition, in both unit dose forms and non-unit dose forms, has an increased shelf-life. The increased shelf-life is evidenced by the absence of yellowing of the detergent composition over an extended period of time. In some instances, the increased shelf life is more than at least a month. In some instances, the increased shelf life is more than at least six months. In some instances, the increased shelf life is more than at least one year.

In some instances, detergent compositions according to the disclosure comprise at least one surfactant. The at least one surfactant is typically present in a detergent composition in an amount from about 5 weight percent to about 70 weight percent of the detergent composition, alternatively in an amount from about 15 weight percent to about 65 weight percent of the detergent composition, and alternatively in an amount from about 20 weight percent to about 50 weight percent of the detergent composition. In some instances, the at least one surfactant is present in a detergent composition in an amount ranging from about 30 weight percent to about 40 weight percent of the detergent composition. In the present disclosure, all weight percents are based on the total weight of the detergent composition unless otherwise specified.

In some instances, the at least one surfactant can be selected from a group including anionic surfactants, non-ionic surfactants, cationic surfactants, semi-polar surfactants, zwitterionic surfactants or combinations thereof. In some instances, the at least one surfactant includes one or more anionic surfactants and one or more non-anionic surfactants. In instances where two or more surfactants are employed, the combination of surfactants can be referred to as a surfactant system.

In some instances, the at least one surfactant is an anionic surfactant. When an anionic surfactant is present, the anionic surfactant is typically present in a detergent composition in an amount from about 2 weight percent to about 35 weight percent of the detergent composition, alternatively in an amount from amount from about 8 weight percent to about 25 weight percent of the detergent composition, and alternatively in an amount from about 15 weight percent to about 20 weight percent of the detergent composition. Non-limiting examples of anionic surfactants that can be employed in the present disclosure include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diyIbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), or combinations thereof. In some instances, various classes of anionic surfactants may be used. In some instances, the anionic surfactant is a linear alkyl sulfonate (LAS) or a linear alkylbenzene sulfonate (LABS). LAS and LABS are water soluble salts between 8 and 22 carbon atoms in the alkyl group. In some instances, suitable LAS and/or LABS compounds may include salts of C₈-C₁₈ alkyl sulfonic acids and salts of C₈-C₁₈ alkylbenzyl sulfonic acids. Suitable in some instances, the anionic surfactant is a linear alkyl ether (or laureth) sulfonate. In some instances, suitable linear alkyl ether sulfonates include a linear C₈-C₁₈ alkyl chain, 4-9 repeating ethylene oxide units, and an anionic head group made up of the sulfonate group and a counter cation. Suitable counter cations for LAS, LABS and linear alkyl ether sulfonates include, but are not necessarily limited to, Na⁺, K⁺, and NH₄⁺. In some instances, the anionic surfactant is sodium or potassium lauryl sulfate or a sodium or potassium lauryl ether sulfate. In some instances, the lauryl sulfate or lauryl ether sulfate is in an amount ranging from about 1 to about 10 wt % of the detergent composition. In some instances, the lauryl sulfate or lauryl ether sulfate is in an amount ranging from about 2 to about 8 wt % of the detergent composition.

In some instances, the at least one surfactant is a nonionic surfactant. When a nonionic surfactant is present, the nonionic surfactant is typically present in a detergent composition in an amount from about 2 weight percent to about 30 weight percent of the detergent composition, alternatively in an amount from amount from about weight percent to about 25 weight percent of the detergent composition, and alternatively in an amount from about 15 weight percent to about 20 weight percent of the detergent composition. Non-limiting examples of nonionic surfactants that can be employed in the present disclosure include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA). In some instances, the nonionic surfactant is but not limited to alkoxylated alcohols, polyoxyalkylene alkyl ethers, polyoxyalkylene alkylphenyl ethers, polyoxyalkylene sorbitan fatty acid esters, polyoxyalkylene sorbitol fatty acid esters, polyalkylene glycol fatty acid esters, alkyl polyalkylene glycol fatty acid esters, polyoxyethylene polyoxypropylene alkyl ethers, polyoxyalkylene castor oils, polyoxyalkylene alkylamines, glycerol fatty acid esters, alkylglucosamides, alkylglucosides, alkylamine oxides, or any combinations thereof.

In some instances, the least one surfactant is a cationic surfactant. When a cationic surfactant is present, the cationic surfactant is typically present in a detergent composition in an amount from about 2 weight percent to about 35 weight percent of the detergent composition, alternatively in an amount from amount from about 8 weight percent to about 25 weight percent of the detergent composition, and alternatively in an amount from about 15 weight percent to about 20 weight percent of the detergent composition. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, or combinations thereof.

In some instances, the detergent composition can include at least one semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, or combinations thereof.

In some instances, the detergent composition can include at least one zwitterionic surfactant. Non-limiting examples of zwitterionic surfactants that can be employed include betaines such as alkyldimethylbetaines, sulfobetaines, or combinations thereof.

When a semipolar surfactant and/or a zwitterionic surfactant are present, each of the semipolar surfactant and/or a zwitterionic surfactant is typically present in a detergent composition in an amount from about 2 weight percent to about 35 weight percent of the detergent composition, alternatively in an amount from amount from about 8 weight percent to about 25 weight percent of the detergent composition, and alternatively in an amount from about 15 weight percent to about 20 weight percent of the detergent composition.

In some instances, the at least one surfactant is a surfactant system that includes a fatty alcohol ethoxylate C₁₂₋₁₅ 7EO (non-ionic surfactant) and sodium laureth sulfate (anionic surfactant).

As stated above, detergent compositions according to the disclosure also include at least one amine source. The amine source is typically present in a detergent composition in an amount from about 5 weight percent to about 30 weight percent of the detergent composition, alternatively in an amount from about 10 weight percent to about 25 weight percent of the detergent composition, and alternatively in an amount from about 12 weight percent to about 20 weight percent of the detergent composition. The amine source that can be employed in the present disclosure includes any compound that contains at least one amine functional group and that can be used in laundry detergent formulations. For example, the amine source can include, but is not limited to, an ethanolamine compound, a chelating agent, amino acid, an amine containing enzyme or any combination thereof. For example, a combination of an amino acid and an ethanolamine compound can be present in the detergent composition.

Ethanolamine compounds contain a group of amino alcohols. Ethanolamine compounds can be used as a surfactant in some detergent compositions. Ethanolamine compounds can aid in the removal of dirt, grease and stains. Illustrative examples of ethanolamine compounds include, but are not limited to, monoethanolamine, diethanolamine, triethanolamine or mixtures thereof. In some instances, the ethanolamine is present in an amount ranging from about 0.5 to about 10 wt % of the detergent composition. In some instances, the ethanolamine is present in an amount ranging from about 1.0 to about 5.0 wt % of the detergent composition.

A chelating agent is a compound containing a ligand (typically organic) that can react with metal ions to form a stable, water-soluble complex. Chelating agents can also be referred to as chelants, chelators or sequestering agents. Chelating agents can have a ring-like center which forms at least two bonds with the metal ion. In some instances, the chelate is any compound that includes at least an amine functional and that can be used in laundry detergent formulations. In some instances, the chelating agent is present in an amount ranging from about 0.1 to about 5 wt % of the detergent composition. In some instances, the chelating agent is present in an amount ranging from about 0.1 to about 2.5 wt % of the detergent composition. Exemplary chelates that can be employed as an amine source include, but are not limited to, tetrasodium iminodisuccinate, ethylenediamine, ethylenediaminetetraacetic (EDTA) or mixtures thereof. In some instances, the chelating agent is N,N-bis(carboxymethyl)-L-glutamic acid tetrasodium salt. Chelating agents are sometimes used as water softeners in detergent compositions. In some instances, the chelating agent is iminodisuccinate (IDS), ethylenediaminetetraacetic acid (EDTA), nitrilotriacetic acid, diethylenetriaminepenta(methylenephosphonic acid), nitrilotris(methylenephosphonic acid), ethylenediamine-N,N′-disuccinic acid (EDDS), hydroxyethylenediaminetriacetic acid (HEDTA), N,N-bis(carboxymethyl)-L-glutamic acid tetrasodium salt, Alanine, N,N-bis(carboxymethyl)-alanine, or other chelating compounds comprising an amine group.

Amino acids are organic compounds that contain both amino and carboxylic functional groups. Exemplary amino acids that can be used as the amine source include, but are not limited to, arginine, histidine, lysine, aspartic acid, glutamic acid, serine, threonine, asparagine, glutamine, cysteine, selenocysteine, glycine, proline, alanine, valine, isoleucine, methionine, phenylalanine, tyrosine, tryptophan or any combination thereof.

In some instances, amine-containing enzymes (i.e., enzymes that containing at least one amine functionality) can also be used as the amine source.

Various detergent compositions according to the disclosure further include water. The amount of water that is present in a detergent composition according to the disclosure can vary depending on whether the detergent composition is in unit dose form, or non-unit dose form. This will be described in greater detail herein.

In some instances, detergent compositions according to the disclosure have pHs ranging from about 6 to about 12. In some instances, detergent compositions according to the disclosure have pHs ranging from about 6.5 to about 10. In some instances, detergent compositions according to the disclosure have pHs ranging from about 7 to about 9.

In addition to the above-mentioned components, detergent compositions according to the disclosure can further include any additional detergent component(s) that is(are) known in the art. Other optional additional detergent components include, for example, bleaching systems, anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, carboxymethylcellulose (CMC), and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, and wicking agents, either alone or in any combination thereof. Any component known in the art for use in detergents may be utilized. The choice of such optional components and the working amounts is well within the skill of the artisan.

In some instances, detergent compositions according to the disclosure can further include well known dye transfer agents, fluorescent whitening agents, soil release polymers, rheology modifiers or any combination thereof. In some instances, detergent compositions according to the disclosure can also include a fatty acid that has a formula R—C(O)OH, wherein R is a C₅-C₂ linear or branched aliphatic group. In one example, the fatty acid is dodecanoic acid (also known as coconut fatty acid). In some embodiments of the present invention, the fatty acid is present in an amount ranging from about 0.1 to about 10 wt % of the detergent composition. In some instances, the fatty acid is present in an amount ranging from about 0.5 to about 5 wt % of the detergent composition.

In some instances, detergent compositions according to various aspects of the disclosure may include an anti-redeposition polymer. In some instances, anti-redeposition agents include polymers with a soil detachment capacity, which are also known as “soil repellents” due to their ability to provide a soil-repelling finish on the treated surface, such as a fibers. In some instances, the anti-redeposition polymer is in an amount ranging from about 0.1 to about 1 wt % of the detergent composition. In some instances, the anti-redeposition polymer is an acrylic/styrene copolymer. In some instances, the polymer can be a polyester. In some instances, the polyesters include co-polyesters prepared from dicarboxylic acids, such as adipic acid, phthalic acid or terephthalic acid. In some instances, an anti-redeposition agents includes polyesters with a soil detachment capacity that include those compounds which, in formal terms, are obtainable by esterifying two monomer moieties, the first monomer being a dicarboxylic acid HOOC-Ph-COOH and the second monomer a diol HO—(CHR—)ₐOH, which may also be present as a polymeric diol H—(O—(CHR—)ₐ)_{b}OH. “Ph” here means an ortho-, meta- or para-phenylene residue that may bear 1 to 4 substituents selected from alkyl residues with 1 to 22 C atoms, sulfonic acid groups, carboxyl groups and mixtures thereof, “R” means hydrogen or an alkyl residue with 1 to 22 C atoms and mixtures thereof, “a” means a number from 2 to 6 and “b” means a number from 1 to 300. In some instances, the polyesters obtainable therefrom may contain not only monomer diol units —O—(CHR—)ₐO— but also polymer dial units —(O—(CHR—)ₐ)_{b}O—. In some instances, the molar ratio of monomer diol units to polymer diol units may amount to from about 100:1 to about 1:100, or alternatively from about 10:1 to about 1:10. In some instances, the polymer diol units, the degree of polymerization “b” may be in the range of from about 4 to about 200, or alternatively from about 12 to about 140. In some instances, the number average molecular weight of the polyesters with a soil detachment capacity may be in the range of from about 250 to about 100,000, or alternatively from about 500 to about 50,000. In some instances, the acid on which the residue Ph is based may be selected from terephthalic acid, isophthalic acid, phthalic acid, trimellitic acid, mellitic acid, the isomers of sulfophthalic acid, sulfoisophthalic acid and sulfoterephthalic acid and mixtures thereof. In some instances, the acid groups thereof are not part of the ester bond in the polymer, they may be present in salt form, such as an alkali metal or ammonium salt.

In some instances, instead of the monomer HOOC-Ph-COOH, polyesters with a soil detachment capacity (the anti-redeposition agent) may include small proportions, for example up to about 10 mole percent relative to the proportion of Ph with the above-stated meaning, of other acids that include at least two carboxyl groups. These include, for example, alkylene and alkenylene dicarboxylic acids such as malonic acid, succinic acid, fumaric acid, maleic acid, glutaric acid, adipic acid, pimelic acid, suberic acid, azelaic acid and sebacic acid. Exemplary diols HO—(CHR—)ₐOH include those in which R is hydrogen and “a” is a number of from about 2 to about 6, and in some instances those in which “a” has the value of 2 and R is selected from hydrogen and alkyl residues with 1 to 10 C atoms, or where R is selected from hydrogen and alkyl residues with 1 to 3 C atoms in another embodiment. Examples of diol components include ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol and neopentyl glycol. The polymeric diols may include polyethylene glycol with an average molar mass in the range from about 1000 to about 6000. In some instances, these polyesters may also be end group-terminated, with end groups that may be alkyl groups with 1 to 22 C atoms or esters of monocarboxylic acids. The end groups attached via ester bonds may be based on alkyl, alkenyl and aryl monocarboxylic acids with 5 to 32 C atoms, or alternatively with 5 to 18 C atoms. These may include valeric acid, caproic acid, enanthic acid, caprylic acid, pelargonic acid, capric acid, undecanoic acid, undecenoic acid, lauric acid, lauroleic acid, tridecanoic acid, myristic acid, myristoleic acid, pentadecanoic acid, palmitic acid, stearic acid, petroselinic acid, petroselaidic acid, oleic acid, linoleic acid, linolaidic acid, linolenic acid, eleostearic acid, arachidic acid, gadoleic acid, arachidonic acid, behenic acid, erucic acid, brassidic acid, clupanodonic acid, lignoceric acid, cerotic acid, melissic acid, benzoic acid, which may bear 1 to 5 substituents having a total of up to 25 C atoms, or alternatively 1 to 12 C atoms, for example tert-butylbenzoic acid. The end groups may also be based on hydroxymonocarboxylic acids with 5 to 22 C atoms, which for example include hydroxyvaleric acid, hydroxycaproic acid, ricinoleic acid, the hydrogenation product thereof, hydroxystearic acid, and ortho-, meta- and para-hydroxybenzoic acid. The hydroxymonocarboxylic acids may in turn be joined to one another via their hydroxyl group and their carboxyl group and thus be repeatedly present in an end group. The number of hydroxymonocarboxylic acid units per end group, i.e. their degree of oligomerization, may be in the range of from 1 to 50, or alternatively in the range of from 1 to 10. In some instances, polymers of ethylene terephthalate and polyethylene oxide terephthalate, in which the polyethylene glycol units have molar weights of from about 750 to about 5000 and the molar ratio of ethylene terephthalate to polyethylene oxide terephthalate of from about 50:50 to about 90:10, can be used alone or in combination with cellulose derivatives.

In some instances, detergent compositions according to various aspects of the disclosure may include an optical brightener. Optical brighteners adsorb ultraviolet and/or violet light and re-transmit it as visible light, typically a visible blue light. Optical brighteners include, but are not limited to, derivatives of diaminostilbene disulfonic acid or alkali metal salts thereof. Suitable compounds are, for example, salts of 4,4′-bis(2-anilino-4-morpholino-1,3,5-triazinyl-6-amino)stilbene 2,2′-disulfonic acid or compounds of similar structure which, instead of the morpholino group, bear a diethanolamino group, a methylamino group, an anilino group or a 2-methoxyethylamino group. Optical brighteners of the substituted diphenylstyryl type may furthermore be present, such as the alkali metal salts of 4,4′-bis(2-sulfostyryl)diphenyl, 4,4′-bis(4-chloro-3-sulfostyryl)diphenyl, or 4-(4-chlorostyryl)-4′-(2-sulfostyryl)diphenyl. Mixtures of the above-stated optical brighteners may also be used. In some instances, optical brighteners may be present in detergent compositions in an amount ranging about 0.01 to about 0.5 wt %, alternatively from about 0.01 to about 1 wt %, alternatively from about 0.01 to about 3 wt %, or alternatively from 0.01 to about 5 wt % of the detergent composition.

In some instances, detergent compositions according to various aspects of the disclosure may include one or more foam inhibitors (i.e., defoamers). In some instances, foam inhibitors include, but are not limited to, fatty acids such as coconut fatty acids. In some instances, suitable foam inhibitors include, for example, soaps of natural or synthetic origin (which may exhibit elevated proportions of C₁₈-C₂₄ fatty acids), organopolysiloxanes and mixtures thereof with microfine (and optionally silanized) silica, paraffins, waxes, microcrystalline waxes and mixtures thereof with silanized silica or bis-fatty acid alkylenediamides (for example, bistearylethylenediamide), silicones, and combinations thereof. In some instances, foam inhibitors may be present in a detergent composition in an amount ranging about 0.01 to about 0.5 wt %, alternatively from about 0.01 to about 0.1 wt %, alternatively from about 0.01 to about 3 wt %, or alternatively from 0.01 to about 5 wt % of the detergent composition.

In some instances, detergent compositions according to various aspects of the disclosure may include bittering agents to hinder accidental ingestion of the composition. Bittering agents are compositions that taste bad, so children or others are discouraged from accidental ingestion. In some instances, the bittering agent is denatonium benzoate, aloin, or others. Bittering agents may be present in the composition in an amount ranging about 0.01 to about 0.5 wt %, alternatively from about 0.01 to about 0.1 wt %, alternatively from about 0.01 to about 3 wt %, or alternatively from 0.01 to about 5 wt % of the detergent composition.

In some instances, detergent compositions according to the disclosure can further include at least one other enzyme besides a Maillard reaction inhibiting enzyme as described above. The at least one other enzyme can enhance the cleaning performance (including, for example, stain removal, redeposition of soil released in the washing/cleaning process, or restoration, fully or partially, the whiteness of a textile) of other components that are present in a detergent composition. Exemplary enzymes other than Maillard reaction inhibiting enzymes include, but are not limited to, deoxyribonucleases (DNaeses), cellulases, proteases, lipases, cutinases, amylases, peroxidases, and/or oxidase. In some instances, the at least one other enzyme besides the Maillard reaction inhibiting enzyme is in an amount ranging from about 0.001 weight percent to about 3.0 weight percent of the detergent composition. In some instances, the Maillard reaction inhibiting enzyme is in an amount ranging from about 0.01 to about 5.0 weight percent of the detergent composition. In some instances, the Maillard reaction inhibiting enzyme is in an amount ranging from about 0.01 to about 5.0 weight percent of a raw composition.

Detergent compositions according to various aspects of the disclosure can be formulated utilizing techniques well known to those skilled in the art. In some instances, the various components that provide a detergent composition according to the disclosure can be added in any order. For example, a formulation according to the disclosure can be added to a vessel including at least one surfactant, water and an amine source. The addition is typically a metered addition. Alternatively, a formulation according to the disclosure can be added to a reactor vessel and thereafter each of the at least one surfactant, water and amine source can be added as a mixture or individually. The addition of the various components that provide a detergent composition according to the disclosure can be performed with continuous stirring. The addition is typically performed at room temperature, e.g., from about 20° C. to about 30° C. In some instances, an enzyme enhancing agent can be added to the during the formulation process to improve the performance of the Maillard reaction inhibiting enzyme. In some instances, more than one Maillard reaction inhibiting enzyme as defined above can be added as a component of a detergent composition according to the disclosure.

In some instances, a detergent composition according to the disclosure is in a unit dose form. In such instances, a detergent product is provided that includes a container and a detergent composition, where the detergent composition comprises, consists essentially of, or consists of at least one surfactant, a formulation according to the disclosure, an amine source that is reactive with a reducing sugar monomer according to the disclosure, and optionally water.

The container may be a pouch, a pod or a pack (or pac) made from a water-soluble or water-dispersible polymer film, which encloses the detergent composition. The water-soluble or water-dispersible container can be in any desirable shape and size, e.g., a square, a rectangular, an oval, an ellipsoid, a super-elliptical, or a circular shape.

As stated above, a container of a unit dose is formed from a water-soluble or water-dispersible polymer film. Non-limiting examples of water-soluble or water-dispersible polymers include, but are not limited to, polyvinyl alcohol, cellulose ethers, polyethylene oxide, starch, polyvinylpyrrolidone, polyacrylamide, polyacrylonitrile, polyvinyl methyl ether-maleic anhydride, polymaleic anhydride, styrene maleic anhydride, hydroxyethylcellulose, methylcellulose, polyethylene glycol, carboxymethylcellulose, polyacrylic acid salts, alginates, acrylamide copolymers, guar gum, casein, ethylene-maleic anhydride resins, polyethyleneimine, ethyl hydroxyethylcellulose, ethyl methylcellulose, hydroxyethyl methylcellulose, film forming cellulosic polymer, polyanhydride, polysaccharide, polyalkylene oxide, cellulose, cellulose ester, cellulose amide, polyvinyl acetate, polycarboxylic acid and salt, polyaminoacid, polyamide, natural gums, polyacrylate, water-soluble acrylate copolymer, methylcellulose, carboxymethylcellulose sodium, dextrin, ethylcellulose, maltodextrin, polymethacrylate, polyvinyl alcohol copolymer, and combinations thereof.

In some instances, the water-soluble or water-dispersible film material of a container may be polyvinyl alcohol, polyvinyl acetate, film forming cellulosic polymer, polyacrylic acid, polyacrylamide, polyanhydride, polysaccharide, or a mixture thereof. In one example, the water-soluble or water-dispersible film material is polyvinyl alcohol or polyvinyl acetate. In another example, the water-soluble or water-dispersible container is made from a lower molecular weight water-soluble polyvinyl alcohol film-forming resin.

In some instances, the water-soluble or water-dispersible container can further contain a cross-linking agent. In some instances, the cross-linking agent can be boric acid or sodium borate.

In some instances, the film material on a container according to the disclosure can have a thickness of between about 50 microns to about 120 microns, alternatively a thickness between about 60 microns to about 100 microns being. In some instances, the film material on a container can have a thickness ranging in an amount from about 30 microns to about 150 microns.

The water-soluble or water-dispersible container of unit dose embodiments can be prepared in any suitable way, such as via molding, casting, extruding or blowing, and is then filled using an automated filling process, as known in the prior art.

Detergent compositions that can be used in a unit dose embodiment include (i) a detergent composition as described herein; and (ii) a solvent system. The solvent system can consist of water, a non-aqueous solvent (NAS), and a residual solvent present in an amount of 0 to 5 percent by weight of the liquid composition. In some instances, the NAS can include a single NAS. In some instances, the NAS can include more than one non-aqueous solvent. The residual solvent is neither water nor the NAS. Each of water and the NAS is present in an amount of more than 5 percent by weight of the detergent composition, with the solvent system totals from about 30% to about 80% by weight of the detergent composition.

In some instances, the solvent system comprises from about 37.5% to about 70%, preferably from about 40% to about 65%, and more preferably from about 50% to about 60%, based on the total weight of the detergent composition. In other instances, the solvent system is present in an amount of from 37.5% to about 40%, from about 40% to about 45%, from about 45% to about 50%, from about 50% to about 55%, from about 55% to about 60%, from about 60% to about 65%, from about 65% to about 70%, from about 70% to about 75%, or from about 75% to about 80%, by weight of the detergent composition.

Typically, two or three non-aqueous solvents which are greater than 5% wt. are present in the liquid composition. In some instances, the NAS is typically present from about 10% to about 70%, more typically from about 20% to about 65%, even more typically from about 25% to about 60%, yet more typically from about 30% to about 55%, and still yet more typically from about 40% to about 55%, based on the total weight of the detergent composition.

In other instances, the NAS is present in an amount of from about 10% to about 20%, from 20% to about 30%, from about 30% to about 40%, from about 40% to about 50%, from about 50% to about 60%, from about 60% to about 70%, by weight of the detergent composition.

In some instances, the NAS is present in an amount of about 25%, from 22% to about 27%, from 28% to about 35%, from about 33% to about 43%, or from about 40% to about 45%, by weight of the detergent composition. In some instances, the NAS is in an amount ranging from about 15 to about 40 wt % of the detergent composition. In some instances, the NAS is in an amount ranging from about 10 to about 50 wt % of the detergent composition.

In some instances, the NAS may be chosen from ethanol, polyethylene glycol; polypropylene glycol; polypropylene glycol esters; polyethylene glycol esters such as polyethylene glycol stearate, polyethylene glycol laurate, and/or polyethylene glycol palmitate; methyl ester ethoxylate; diethylene glycol; dipropylene glycol; sorbitol; tetramethylene glycol; butylene glycol; pentanediol; hexylene glycol; heptylene glycol; octylene glycol; 2-methyl-1,3-propanediol; xylitol; mannitol; erythritol; dulcitol; inositol; adonitol; triethylene glycol; glycol ethers, such as ethylene glycol monobutyl ether, diethylene glycol monobutyl ether, triethylene glycol monobutyl ether, ethylene glycol monopropyl ether, diethylene glycol monoethyl ether, triethylene glycol monoethyl ether, diethylene glycol monomethyl ether, and triethylene glycol monomethyl ether; tris(2-hydroxyethyl)methyl ammonium methylsulfate; ethylene oxide/propylene oxide copolymers with the non-aqueous solvent has a weight average molecular weight of 4000 Daltons or less.

In some instances, the NAS is selected from polyethylene glycol; polyethylene glycol esters such as polyethylene glycol stearate, polyethylene glycol laurate, and/or polyethylene glycol palmitate; and polypropylene glycol.

In some instances, the NAS is glycerol (glycerin), ethylene glycol, ethanol, or a 4C+ compounds. The term “4C+ compound” refers to one or more of: polyethylene glycol esters such as polyethylene glycol stearate, propylene glycol laurate, and/or propylene glycol palmitate; ethyl ester ethoxylate; diethylene glycol; dipropylene glycol; tetramethylene glycol; butylene glycol; pentanediol; hexylene glycol; heptylene glycol; octylene glycol; 2-methyl-1,3-propanediol; triethylene glycol; glycol ethers, such as ethylene glycol monobutyl ether, diethylene glycol monobutyl ether, triethylene glycol monobutyl ether, ethylene glycol monopropyl ether, diethylene glycol monoethyl ether, triethylene glycol monoethyl ether, diethylene glycol monomethyl ether, and triethylene glycol monomethyl ether; tris(2-hydroxyethyl)methyl ammonium methylsulfate; ethylene oxide/propylene oxide copolymers with a number average molecular weight of 3,500 Daltons or less; and ethoxylated fatty acids. In some instances, the non-aqueous solvent is or includes a relatively low molecular weight polyethylene glycol (PEG). In some instances, the polyethylene glycol has a molecular weight ranging from about 200 to about 1000 g/mol (or Daltons), alternatively a molecular weight ranging from about 200 to about 800 g/mol, or alternatively a molecular weight of about 400 g/mol. In some instances, the polyethylene glycol has a weight average molecular weight of less than about 600 Da, e.g. about 400, such as those having a weight average molecular weight of from about 380 to about 420 Da. In other instances, PEG 200, PEG 250, PEG 300, PEG 350, PEG 400, PEG 450, PEG 500, PEG 550, and/or PEG 600 (wherein the numerals represent the approximate weight average molecular weight in Daltons or grams/mol) may be used. In some instances, the non-aqueous solvent is or includes an ethylene oxide/propylene oxide block copolymer. In some instances, the NAS is or includes a polyol such as glycerin. In some instances, the non-aqueous solvent is or includes a mixture of a polyol and a polyethylene glycol.

In some instances, the polyol in the mixture is glycerin. Suitable polyol/polyethylene glycol mixtures may have a polyol to polyethylene glycol weight:weight ratio ranging from about 10:1 to about 1:10, alternatively from about 9:1 to about 1:9, alternatively from about 8:1 to about 1:8, alternatively from about 7:1 to about 1:7, alternatively from about 6:1 to about 1:6, alternatively from about 5:1 to about 1:5, alternatively from about 4:1 to about 1:4, alternatively from about 3:1 to about 1:3, alternatively from about 2:1 to about 1:2, alternatively from about 1.5:1 to about 1:1.5, and alternatively about 1:1

In instances, the NAS is polyethylene glycol (“PEG”) and/or an ester thereof. The PEG can have a weight average molecular weight ranging, for example, from about 100 to about 4000 Daltons. Suitable PEGs can have a weight average molecular weight of, for example, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, or about 2600, about 2700, about 2800, about 2900, about 3000, about 3500, or about 4000 Daltons. Suitable polyol/polyethylene glycol mixtures may have a polyol to polyethylene glycol weight:weight ratio ranging from about 10:1 to about 1:10, alternatively from about 9:1 to about 1:9, alternatively from about 8:1 to about 1:8, alternatively from about 7:1 to about 1:7, alternatively from about 6:1 to about 1:6, alternatively from about 5:1 to about 1:5, alternatively from about 4:1 to about 1:4, alternatively from about 3:1 to about 1:3, alternatively from about 2:1 to about 1:2, alternatively from about 1.5:1 to about 1:1.5, and alternatively about 1:1.

In some instances, the NAS is PEG-100 stearate, PEG-400, or PEG-3350. In other instances, the NAS is PEG-400 in an amount of from about 20% to about 45% by weight of the detergent composition; while water is present in an amount of from about 10% to about 30% by weight of the detergent composition.

The water in the detergent composition of the unit-dose form can be derived from added water or water accompany a component that forms the raw material that is used in formulating the detergent composition. The total water amount presented in the detergent composition is typically from about 5% to about 45%, more typically from about 10% to about 40%, even more typically from about 15% to about 35%, still more typically from about 20% to about 40%, yet more typically from about 25% to about 35%, and even further typically from about 25% to about 30%, based on the total weight of the detergent composition.

In other instances, water is present in an amount of from about 5% to about 10%, from 10% to about 15%, from about 15% to about 20%, from about 20% to about 25%, from about 25% to about 30%, from 30% to about 35%, from about 35% to about 40%, or from about 40% to about 45%, by weight of the detergent composition.

In some instances, water is present in an amount of from 11% to about 16%, from 17% to about 23%, or from about 22% to about 32%, by weight of the detergent composition.

In some instances, the weight ratio of water to the NAS is from 1:4 to 4:1, from 1:3 to 3:1, from 1:3 to 2:1, from 1:2 to 2:1, or about 1:1. In other embodiments, the weight ratio of water to the NAS is from 1:1 to 4:1, from 1:1 to 3:1, or from 1:1 to 2:1. In further embodiments, the weight ratio of water to the NAS is about 0.5:1, about 0.8:1, about 1:1, about 1:1, about 1.2:1, about 1.3:1, or about 1.5:1.

The term “residual solvent(s)” generally refers to a solvent that is introduced into a detergent composition by the addition of an ingredient (i.e., a commercial product containing the ingredient and the residual solvent), where the residual solvent is less than 5% by weight of the detergent composition. In some instances, the residual solvent is less than 5% wt, or less than 3% wt, or less than 1% wt.

The residual solvent is neither water nor the NAS. In some instances, the residual solvent is a mono-ol or di-ol with a low molecular weight. For example, the residual solvent may be ethanol. The existence of the residual solvent does not interfere with the performance of the solvent system.

As mentioned above, detergent compositions according to the disclosure can be encapsulated in a container made of a water-soluble or water-dispersible film. The solubility of the polymeric film in water should be moderated to keep the film structurally sound prior to use. In some instances, the inclusion of a non-aqueous solvent of certain type in the detergent composition moderates the solubility of the film, thereby protecting the film from being dissolved by water incorporated in the detergent composition. As such, adding the non-aqueous solvent to the detergent composition allows for unit dose packs where the detergent composition therein includes water present in amounts of up to about 45%, by the weight of the detergent composition. It also allows for unit dose packs where the detergent composition therein includes contain a high total solvent content, up to about 80%, by the weight of the detergent composition without compromising pack rigidity.

In some instances, a detergent composition according to the disclosure is in a liquid detergent form (i.e., non-unit dose form). In such instances, the detergent composition can comprise, consist essentially of, or consist of at least one surfactant, a formulation according to the disclosure, an amine source that can react with the reducing sugar monomer, and water. In such instances, the detergent composition can contain at least 20 percent by weight and up to 95 percent by weight water, such as up to about 70 percent by weight water, up to about 65 percent by weight water, up to about 55 percent by weight water, up to about 45 percent by weight water, up to about 35 percent by weight water. Other types of solvents, besides water including, for example, organic solvents comprising alkanols, amines, diols, ethers and/or polyols can also be included in a non-unit dose liquid detergent composition according to the disclosure. The organic solvent can be present in a liquid detergent composition according to the disclosure in an amount from about 0 percent by weight, up to, and including, 30 percent by weight.

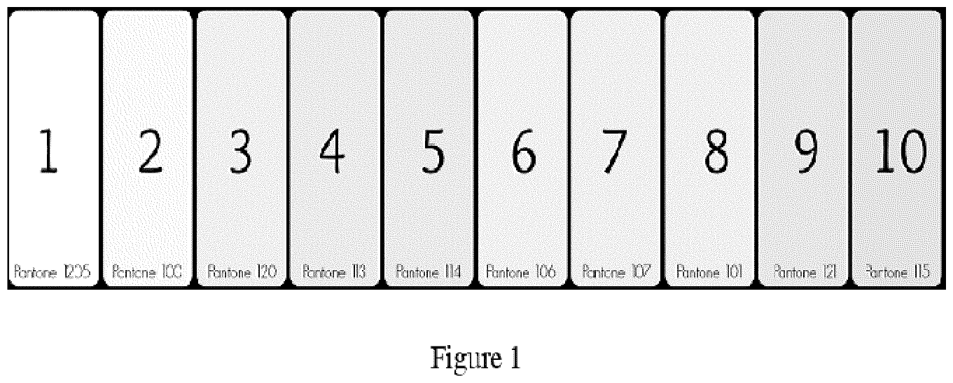

In accordance with various aspects of the disclosure, the yellowing in a detergent composition, such as a liquid laundry detergent composition, can be measured through the pantone color systems. FIG. 1 displays the numerical representation of each color of yellow. Table 1 is also the tabular representation of FIG. 1.

TABLE 1

| Numerical Representation | Pantone Number |
|---|---|
| 1 | Pantone 1205 |
| 2 | Pantone 100 |
| 3 | Pantone 120 |
| 4 | Pantone 113 |
| 5 | Pantone 114 |
| 6 | Pantone 106 |
| 7 | Pantone 107 |
| 8 | Pantone 101 |
| 9 | Pantone 121 |
| 10 | Pantone 115 |

Examples have been set forth below for the purpose of further illustrating the present disclosure. The scope of the present disclosure is not limited to any of the examples set forth herein.

### Example 1

Formulations were prepared by mixing the components in the amounts listed in Table 2. Notably, formulations as tabulated in Table 1 were batched using a standard overhead mixer, target pH 7.5 to 8. Formula 1 and Formula 2 of Table 2 are identical in composition except that Formula 2 included a Maillard reaction inhibiting enzyme as described herein, while no such enzyme was used in preparing Formula 1. Formula 1 is a comparative example (namely CEl), while Formula 2 is an example in accordance with an embodiment of the present disclosure.

TABLE 2

| | Raw | Wt % of | Wt % of |
|---|---|---|---|
| | Material | Formula 1 | Formula 2 |
| Component | Activity | (CE1) | (Inventive) |
| Sorbitol | 100 | 70 | 70 |
| Reducing Sugar Content | 100 | 0.3 or less | 0.3 or less |
| leftover from Glucose to | | | |
| Sorbitol conversion | | | |
| Water | 100 | 28.5 to 31.5 | 28.5 to 31.5 |
| | | (i.e. QS to 100) | (i.e. QS to 100) |
| Diethylene Glycol and | 100 | 0.2% or less | 0.2% or less |
| Ethylene Glycol Impurities | | | |
| Inorganic Impurities such | 100 | 1 ppm or less | 1 ppm or less |
| as Nickel | | | |
| Maillard reaction | 100 | 0 | 0.01 to 1.5 |
| inhibiting enzyme | | | |
| Total | | 100 | 100 |

Formulations were prepared by mixing the components in the amounts listed in Table 3. Notably, formulations as tabulated in Table 3 were batched using a standard overhead mixer, target pH 7.5 to 8. Formula 3 and Formula 4 of Table 3 are identical in composition except that Formula 4 included a Maillard reaction inhibiting enzyme as described herein, while no such enzyme was used in preparing Formula 3. Formula 3 is a comparative example (namely CE3), while Formula 4 is an example in accordance with an embodiment of the present disclosure.

TABLE 3

| | | Wt % of | Wt % of |
|---|---|---|---|
| | Raw Material | Formula 3 | Formula 4 |
| Component | Activity | (CE2) | (Inventive) |
| Starch-based Sustainable | 100 | 20 to 50 | 70 |
| polymer for detergents | | | |
| Reducing Sugar Content | 100 | 1 or less | 1 or less |
| leftover from Glucose to | | | |
| Sorbitol conversion | | | |
| Water | 100 | QS to 100 | QS to 100 |
| Acid such as Citric, if | 100 | 1% or less | 1% or less |
| needed to lower pH into | | | |
| preservation pH range | | | |
| Preservative such as BIT, | 100 | 0.5% or less | 0.5% or less |
| CIT and or MIT | | | |
| Maillard reaction | 100 | 0 | 0.01 to 1.5 |
| inhibiting enzyme | | | |
| Total | | 100 | 100 |

Pre-Mix Formulations 5 and 6 were prepared by mixing components in the amount listed in Table 4. Notably, Pre-Mix Formulations 5 and 6 were batched using a standard overhead mixer for 1 hour. Glucose was added to the batch to illustrate the inclusion of a reducing sugar in the sustainable material (for example, sorbitol, which may exhibit lot-to-lot variation in the % of residual glucose).

TABLE 4

| | | Formula 5 | Formula 6 |
|---|---|---|---|
| Ingredient | Active % | (wt %) | (wt %) |
| Water | 100 | 82.5% | 82.7% |
| Sorbitol | 70 | 16.9% | 16.9% |
| Glucose | 100 | 0.3% | 0.3% |
| Hexose Oxidase | 100 | 0.2% | 0.0% |

Formulas 7 and 8 were created with Pre-Mix Formulations 5 and 6, respectively, and then were placed in a stability chamber at 25° C. or 37° C. as seen in Table 5. Yellow color was observed after 1 month of stability. Pre-Mix Formulation 5, which includes the enzyme Hexose Oxidase, showed a color benefit over Pre-Mix Formulation 6, which did not include Hexose Oxidase. Results of the yellow coloring observed are depicted in Table 6.

TABLE 5

| Ingredient | Active % | Wt % | Wt % |
|---|---|---|---|
| Pre-Mix of Formulation 5 | N/A | 29.5 | 0 |
| Pre-Mix of Formulation 6 | N/A | 0 | 29.5 |
| Glycerin | 99 | 25 | 25 |
| Alcohol Ethoxylate 25-7 | 100 | 35 | 35 |
| Ethanol | 100 | 2.5 | 2.5 |
| Fatty Acid | 100 | 4 | 4 |
| MEA | 100 | 1 | 1 |
| QS/additional MEA | 100 | 3 | 3 |
| Total | | 100 | 100 |

TABLE 6

| Formulas | Pantone Color Ranking (0-10)* |
|---|---|
| Formula 7 (1 month at 25 C.) | 0 |
| Formula 8 (1 month at 25 C.) | 1 |
| Formula 7 (1 month at 37 C.) | 5 |
| Formula 8 (1 month at 37 C.) | 10 |

While the present disclosure has been particularly shown and described with respect to preferred embodiments thereof, it will be understood by those skilled in the art that the foregoing and other changes in forms and details may be made without departing from the spirit and scope of the present disclosure. It is therefore intended that the present disclosure not be limited to the exact forms and details described and illustrated, but fall within the scope of the appended claims.

What is claimed is:

1. A raw material composition comprising:
one or more reducing sugars in an amount of from about 0.01 to about 5 wt % of the raw material composition;
a sustainable raw material in an amount of from about 1 to about 99 wt % of the raw material composition; and
a Maillard reaction inhibiting enzyme in an amount of from about 0.01 to about 5 wt % of the raw material composition.

2. The raw material composition of claim 1, wherein the sustainable raw material is selected from the group consisting of a starch, a glycogen, a galactogen, a cellulose, a chitin, and any mixture thereof.

3. The raw material composition of claim 1, wherein the sustainable raw material is selected from the group consisting of sorbitol, xylitol, mannitol, lactitol, isomalt, maltitol, a hydrogenated starch hydrolysate, sucrose, stachyose, verbascose, trehalose, raffinose, and any combination thereof.

4. The raw material composition of claim 1, wherein the sustainable raw material comprises:
a sustainable polymer,
a sustainable solvent, or
a mixture of a sustainable polymer and a sustainable solvent.

5. The raw material composition of claim 1, wherein the one or more reducing sugars are selected from the group consisting of fructose, glucose, hexose, lactose, glyceraldehyde, arabinose, maltose, galactose, ribose, xylose, cellobiose, and any combination thereof.

6. The raw material composition of claim 1, wherein the Maillard reaction inhibiting enzyme is selected from the group consisting of hexose oxidase, fructosamine oxidase, fructosamine kinase, carbohydrate oxidase, and any combination thereof.

7. A detergent composition comprising:
a Maillard reaction inhibiting enzyme in an amount of from about 0.01 to about 5 wt % of the detergent composition;
a non-aqueous solvent in an amount of from about 20 to about 50 wt % of the detergent composition;
an alcohol ethoxylate non-ionic surfactant in an amount of from about 15 to about 40 wt % of the detergent composition;
an ethanol amine in an amount of from about 1.0 to about 5.0 wt % of the detergent composition;
at least one reducing sugar in an amount of from about 0.01 to about 5 wt % of the detergent composition; and
water.

8. The detergent composition of claim 7, wherein the Maillard reaction inhibiting enzyme is selected from the group consisting of hexose oxidase, fructosamine oxidase, fructosamine kinase, carbohydrate oxidase, and any combination thereof.

9. The detergent composition of claim 7, wherein the at least one reducing sugar is selected from the group consisting of fructose, glucose, hexose, lactose, glyceraldehyde, arabinose, maltose, galactose, ribose, xylose, cellobiose, and any combination thereof.

10. The detergent composition of claim 7, wherein the non-aqueous solvent is selected from the group consisting of glycerin, a polyethylene glycol, a glycol ether, and any combination thereof.

11. The detergent composition of claim 7, wherein the detergent composition further comprises at least one of:
a base in an amount of from about 0.01 to about 1 wt % of the detergent composition;
an acid in an amount of from about 0.01 to about 1 wt % of the detergent composition;
a chelating agent in an amount of from about 0.1 to about 2.5 wt % of the detergent composition;
a lauryl sulfate or a lauryl ether sulfate in an amount of from about 2 to about 8 wt % of the detergent composition; or
a fatty acid in an amount of from about 0.5 to about 5 wt % of the detergent composition.

12. The detergent composition of claim 7, wherein the detergent composition has a pH of from about 6.5 to about 10.

13. The detergent composition of claim 7, wherein the detergent composition further comprises sorbitol in an amount of from about 10 to about 20 wt % of the detergent composition.

14. A method of preventing browning of a raw material composition, the method comprising:
adding a Maillard reaction inhibiting enzyme to a raw material composition, the raw material composition comprising a sustainable raw material including a reducing sugar, wherein the Maillard reaction inhibiting enzyme prevents the reducing sugar from reacting with an amine source.

15. The method of claim 14, wherein the reducing sugar is selected from the group consisting of fructose, glucose, hexose, lactose, glyceraldehyde, arabinose, maltose, galactose, ribose, xylose, cellobiose, and any combination thereof.

16. The method of claim 14, wherein the Maillard reaction inhibiting enzyme is selected from the group consisting of hexose oxidase, fructosamine oxidase, fructosamine kinase, carbohydrate oxidase, and any combination thereof.

17. The method of claim 14, wherein the sustainable raw material comprises:
a sustainable polymer;
a sustainable solvent; or
a mixture of a sustainable polymer and a sustainable solvent.

18. The method of claim 14, wherein the sustainable raw material is a sustainable polymer, and the sustainable polymer comprises at least one of a starch, a glycogen, a galactogen, a cellulose, a chitin, or any combination thereof.

19. The method of claim 14, wherein the sustainable raw material is a sustainable solvent, and the sustainable solvent comprises sorbitol, xylitol, mannitol, lactitol, isomalt, maltitol, a hydrogenated starch hydrolysate, sucrose, stachyose, verbascose, trehalose, raffinose, or any combination thereof.
